(19) United States
(12) Patent Application Publication
(10) Pub. No.: US 2012/0137414 A1
(43) Pub. Date: Jun. 7, 2012

(54) PEEL-AWAY MEMBRANE STACK FOR POSTERIOR SURFACE OF A LENS

(75) Inventor: Ryan Saylor, Mission Viejo, CA (US)

(73) Assignee: Oakley, Inc., Foothill Ranch, CA (US)

(21) Appl. No.: 12/960,083

(22) Filed: Dec. 3, 2010

(51) Int. Cl. A61F 9/02; B32B 3/02

(52) U.S. Cl. 2/435; 428/80

(57) ABSTRACT

The present invention relates generally to anti-fogging systems used in eyewear, and more particularly to an eyewear system with a stack of peel away membranes positioned on the posterior surface of a goggle lens.

### BACKGROUND OF THE INVENTION

1. Field of the Invention

The present invention relates generally to lenses used in eyewear, and more particularly to a lens system with a stack of membranes positioned posterior to or on the posterior surface of a lens.

2. Description of the Related Art

A wide variety of improvements have been made in recent years in the eyewear field, particularly with respect to eyewear intended for use in active sports. Protective helmet shields or visors are known for such activities as motorcycle riding, football, lacrosse, hockey and the like. Goggles are used for various activities, including skiing, motocross, underwater diving masks, and a variety of industrial safety applications such as welding and for power equipment operators. Typically, goggles offer sealed protection to the eyes and adjacent areas of the wearer's face against particulate matter or water, without providing full head protection.

Conventional eyewear, particularly helmet shields and goggles, are subject to fogging, affecting the vision of the user. Because the wearer's face is usually warmer than the surrounding atmosphere for many applications (particularly for skiing and diving), the lens is often colder than air trapped between the wearer's face and the lens. Warm moisture expressed from the wearer's face tends to increase in concentration within the enclosed space to the point where it condenses upon the inside surface of the lens. Indeed, in extremely cold conditions, as often encountered in skiing applications, condensed moisture can even freeze upon the lens, clouding vision considerably.

To address this problem, some conventional eyewear designs include various ventilation systems that vent moisture from the entrapped air volume on the wearer's side of the lens through the goggle or helmet frame to the external environment. Passive ventilation systems include openings in the eyewear lens and/or frames, but may not provide sufficient airflow to prevent excessive condensation under many conditions. Passive ventilation may also allow wind, snow, hard ice particles, etc. to pass through ventilation openings in the eyewear, compromising the protective purpose. Screens, open cell foams and baffles positioned in or across the vents can preserve the protective function, but at the expense of impaired air flow. Active ventilation systems, such as miniature fans and the like, provide greater airflow, but add power requirements and weight to the eyewear, and are subject to mechanical failure. Other conventional anti-condensation systems for eyewear, such as electrical defoggers, have similar power, weight and reliability issues.

Another proposed solution to the problem of eyewear fogging is to provide additional thermal insulation between the lens surface closest to the wearer's face and the outside atmosphere. Double lens structures, having spaced inner and outer lenses, provide such insulation in many conventional goggle and helmet designs. However, such lenses may still be subject to condensation over time or in extreme environmental or high exertion conditions, impairing or obscuring the field of view.

### SUMMARY OF THE INVENTION

There is provided in accordance with one aspect of the present invention, an anti-fogging system for goggles. The system comprises a goggle, having a lens with a posterior surface which faces a wearer in the as worn orientation. An air space is defined between the posterior surface of the lens and the wearer's face.

At least one transparent membrane removably covers at least a portion of the posterior surface, so that the condensation accumulating on the posterior surface of the membrane can be cleared by removing the membrane from the lens.

Preferably, the system comprises a stack of at least about 5 peel away membranes on the posterior surface of the lens. The system may further comprise a support, for carrying the membranes. The membranes may be removably held together by an adhesive, and may comprise acetate.

There is provided in accordance with a further aspect of the present invention, a method of managing fogging in a goggle. The method comprises the steps of providing a goggle, having a lens with a posterior surface which faces a wearer in the as worn orientation, to define an air space between the posterior surface of the lens and the wearer's face. At least one transparent membrane is removably carried by at least a portion of the posterior surface of the lens.

The goggle is worn until condensation is visible on the membrane, and the membrane is thereafter removed from the goggle to restore vision through the lens.

There is provided in accordance with a further aspect of the present invention, a method of preparing a goggle. The method comprises the steps of providing a goggle having a lens with a posterior surface which faces a wearer in the as worn orientation, to define an air space between the posterior surface of the lens and the wearer's face. At least one transparent membrane is removably secured to the posterior surface. Preferably, at least about 5 transparent membranes are removably secured on the posterior surface.

There is provided in accordance with a further aspect of the present invention, a membrane stack, for attachment to the posterior surface of an eyewear lens. The stack comprises at least three optically transmissive membranes, releasably held together such that a single membrane may be peeled apart from the adjacent membrane. The stack comprises a left optical zone configured for placement in a wearer's left eye line of sight, a right optical zone configured for placement in a wearer's right eye line of sight, and a peripheral edge having a concavity for accommodating a nose region of the eyewear.

The membranes may be releasably held together by an adhesive. Preferably, the membrane stack comprises at least about five membranes, and, in some implementations, at least about ten membranes. At least one of the membranes comprises a pull tab. In some embodiments, a plurality of membranes each comprise a pull tab, and each pull tab may be offset from an adjacent pull tab along an edge of the membrane stack.

The membrane stack may be provided with a preset curvature, such that the membrane stack conforms to a portion of the surface of a sphere or a toroid or other non-cylindrical non-planar geometry.

In accordance with a further aspect of the present invention, there is provided a goggle. The goggle comprises a frame, configured to support a lens in a wearer's field of view and to define an air volume between the lens and the wearer's face in the as worn orientation. A lens is supported by the frame, the lens having a posterior side. At least one connector is provided, for releasably receiving a stack of peel away membranes on the posterior side of the lens. In some embodiments, at least two connectors are provided. The connectors may comprise a post for receiving an aperture on the peel away membrane.

Further features and advantages of the present invention will become apparent from the detailed description of preferred embodiments which follows, when considered together with the attached drawings and claims.

### BRIEF DESCRIPTION OF THE DRAWINGS

These and other aspects of the invention will be readily apparent from the following description and from the appended drawings (not necessarily to scale), which are meant to illustrate and not to limit the invention, and in which:

FIG. 1 is a front and right perspective view of an embodiment of a membrane positioned on the posterior surface of a lens for a goggle.

FIG. 2 is a front schematic elevational view of the membrane and goggle shown in FIG. 1.

FIG. 3 is a side schematic elevational view of the membrane and goggle shown in FIG. 1.

FIG. 4 is top plan view of the membrane and goggle shown in FIG. 1.

FIG. 5 is a bottom plan view of the membrane and goggle of FIG. 1.

FIG. 6 is a side cross-sectional and partially exploded view of a stack of membranes and the goggle taken along lines 6-6 of FIG. 2.

FIG. 7 shows a close-up, partial side cross-sectional view of a single membrane within the membrane stack shown in FIG. 6.

FIG. 8 shows a side cross-sectional and partially exploded view of an embodiment of a membrane stack.

FIG. 9 shows a front schematic view of an embodiment of a membrane stack insert.

FIG. 10 shows a side cross-sectional view of an embodiment of a stack insert taken along lines 9-9 of FIG. 9.

FIG. 11 shows a partial side cross-sectional view of an embodiment of a stack insert taken along lines 9-9 of FIG. 9.

FIG. 12 shows a partial side cross-sectional view of an embodiment of a stack insert taken along lines 9-9 of FIG. 9.

FIG. 13 shows a partial side cross-sectional view of an embodiment of a stack insert taken along lines 9-9 of FIG. 9.

FIG. 14 shows a front elevational view of an embodiment of a stack insert.

FIG. 15 shows a front elevational view of an embodiment of a stack insert.

FIG. 16 shows a partial side view of an embodiment of a membrane stack.

FIG. 17 shows a front top perspective view of an embodiment of a membrane stack.

FIG. 18 shows a side cross-sectional view of another embodiment of a membrane stack proximate to a goggle taken along lines 6-6 of FIG. 2.

### DETAILED DESCRIPTION OF THE DRAWINGS

Although preferred embodiments will be discussed below in terms of double (front-back) lens goggles particularly adapted for snow skiing or other winter sport applications, it will be understood that the invention can also be employed with other eyewear applications, including other types of goggles, shields, helmet shields, military shields or other products which have a closed or partially closed volume of air in the optical path which has a risk of fogging the posterior surface of the lens. Furthermore, while the illustrated embodiments exhibit particular lens surface geometries, front elevational shapes and orientations advantageous to the skiing application, the skilled artisan will readily be able to adapt the membrane stacks disclosed herein to lenses having different geometries and orientations in the as-worn position beyond those illustrated herein. For example, the membrane stack of the present invention can be incorporated into eyewear with a single unitary lens, or dual (right/left) lenses with or without passive or active ventilation, and other known eyewear configurations.

In general, the peel away stack of the present invention may be utilized on any optically transmissive or reflective surface which, due to environmental conditions, experiences deteriorating optical transmission or reflection which can be refreshed by removing an outer layer. In its preferred applications, the peel away membrane stack of the present invention is applied to the posterior surface of an optical window such as an eyeglass lens, which, when mounted in its intended use orientation encloses or partially encloses an air space between the lens and the wearer's face. If the goggle is worn under conditions which cause fogging, (e.g. the accumulation of humidity from the wearer's face exceeds the rate of dissipation through active or passive ventilation, or the drop in temperature of the lens drops below the dew point at any given humidity) the wearer may remove the goggle, peel away the posterior most layer, and replace the goggle now providing a clear field of view. The peel away stack may alternatively be mounted on the anterior surface of the lens or both the posterior and anterior surfaces, depending upon the intended surrounding environmental conditions.

Conventional eyewear may include polarized and/or tinted lenses that provide further protection and comfort to the eyes of the user. However, the nature of the polarization or degree of tint in such eyewear is generally permanently built into a given article of eyewear. Unfortunately, the nature of the desired tint or other optical characteristic may vary depending on the environmental conditions in which the eyewear is being used, or on the particular preference of the user. The membrane stack in accordance with the present invention is optionally configured to allow a user to adjust the amount or presence of its polarization and/or tint or other optical characteristic by incorporating the desired characteristic into one or more peel away membranes as is discussed in additional detail below.

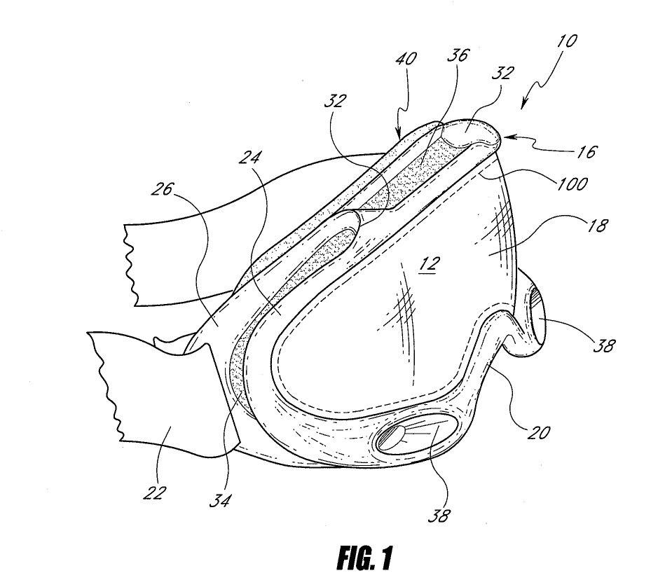

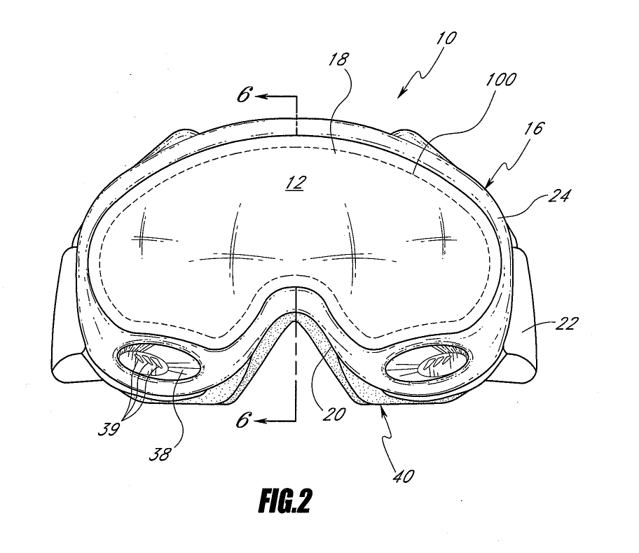

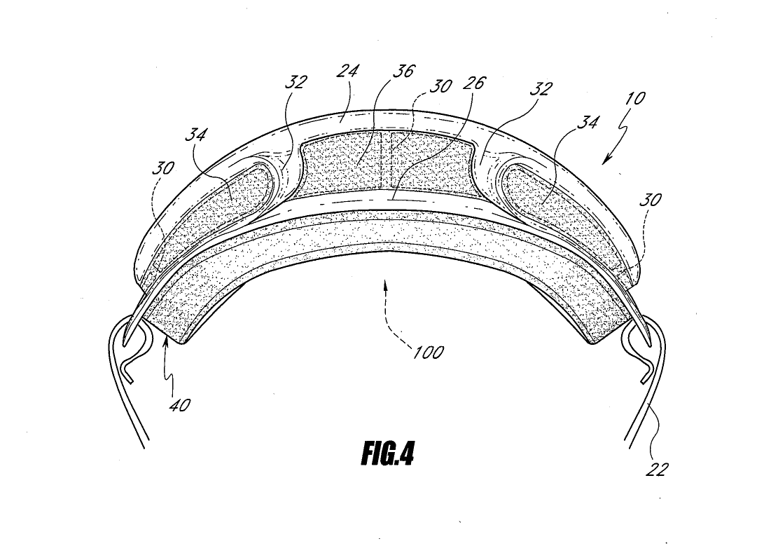

FIGS. 1 to 5 illustrate various views of an embodiment of goggle 10 having at least one removable membrane 100 positioned proximate to or on the posterior surface of a lens. FIG. 6 illustrates a side cross-sectional partially exploded view of a plurality of membranes 100, stacked to form a membrane stack 110, and positioned between the wearer's face and the lens taken along lines 6-6 of FIG. 2. The illustrated membranes are greatly enlarged for clarity. Goggle 10 can comprise a single unitary lens construction, or can comprise one or more lenses, such as the illustrated double unitary lens construction. Goggle 10 can comprise a first, front or outer lens 12, as shown in FIGS. 1 to 3. In some configurations, the goggle 10 can further comprise a second, rear, posterior or inner lens 14, with an inter lens spacing 13 between the outer lens 12 and inner lens 14, as shown in FIG. 6. Outer lens 12 can be separated from the inner lens 14 in any of many different ways known in the art, such as a continuous gasket 15 around the posterior perimeter of the outer lens 12 and the anterior perimeter of the inner lens 14. Each of the illustrated lenses 12, 14 are unitary lenses, configured to extend across the vision of both eyes in an as-worn orientation upon a wearer's head, although other known configurations such as dual side by side lens systems, with or without separate orbitals, may be used with the membrane stack of the present invention.

The illustrated goggle 10 includes a mounting frame 16, configured to support the lenses 12 and 14 on the wearer's head in the as-worn orientation. The illustrated frame 16 can extend continuously around the edges of the lenses, surrounding and defining a unitary viewing window 18 (best seen from FIG. 2) across which the lenses 12 and 14 extend. The bottom edge of the frame 16 can include a medial recess 20 shaped to accommodate the wearer's nose when worn. While such a configuration is preferred for ski goggles, it is not essential for providing many of the advantages of the embodiments of membranes and membrane stacks described herein. In other arrangements, a frame may bound only the bottom edges of the lenses or only the top edges. Alternatively, a frame can bound any other portions of the lenses as will be evident to those of skill in the art. Frameless eyewear can also be constructed in accordance with certain aspects of the present invention. Alternatively, the frame may comprise a helmet, with one or more face shields, e.g., wherein lenses 12 and 14 can comprise an outer and inner helmet shield to cover the front opening in said helmet. In the illustrated embodiment, though, the lens edges can be continuously bounded by the frame 16 of the goggle 10 as shown. The bottom edge of the frame 16 can include a medial recess 20 shaped to rest upon the wearer's nose when worn.

The frame 16 can comprise any of a variety of metals, composites or relatively rigid, molded thermoplastic materials which are well known in the art, and may be substantially transparent, translucent or opaque, or any of a variety of colors. The illustrated frame 16, however, can be preferably constructed of a relatively flexible yet elastic material, which can deform to facilitate application to the wearer's head and return to the original molded shape to retain the lenses 12 and 14 in the desired orientation. Flexibility also facilitates a customized fit to the wearer's face. An exemplary frame material comprises urethane, though other polymeric materials are known which can satisfy these needs. The frame 16 can be manufactured by injection molding, machining and other construction techniques well known in the art.

Referring again to FIGS. 1-5, a strap 22 can be attached to each lateral side of the frame 16, such as by attachment to outriggers in the form of lever arms that may be pivotably attached to the frame. Alternatively, the strap may be looped through slots provided at the lateral edges of the frame 16. In some configurations, the strap 22 can be adjustable and can comprise an elastic material to facilitate stretching over the wearer's head, and to facilitate pulling the frame 16 to conform against the wearer's face. It will be understood that, in other arrangements, the frame can be supported upon the wearer's head by multiple straps, by suspension from a helmet, or by a pair of earstems. Earstems can alternatively attach directly to the lenses or frames in a known fashion.

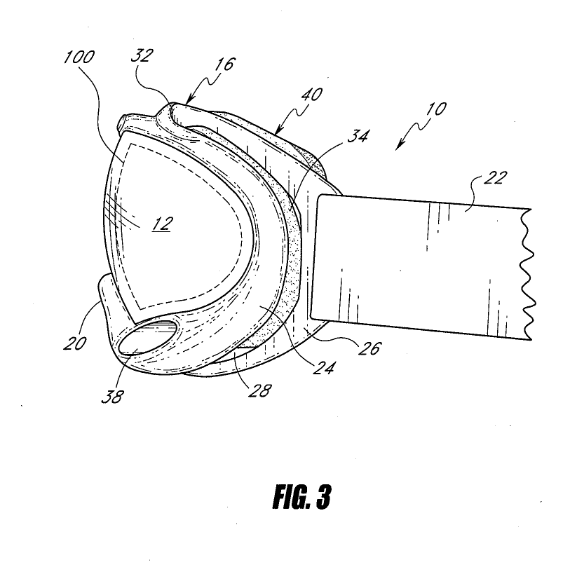

Frame 16 can have a double frame construction, including an outer or front frame portion 24, typically carrying the lenses 12 and 14, and an inner or rear frame portion 26 spaced behind the front frame portion 24 by a plurality of struts. The spacing can taper slightly from a maximum at the top of the goggle to a minimum at the bottom of the goggle 10. Bottom vents 28 are provided to the left and to the right of the medial recess 20. Each of the vents 28 can be upwardly recessed relative to the bottom surfaces of the front frame portion 24 and the rear frame portion 26. The illustrated frame portions 24, 26 can also be connected to one another by a one or more thin ribs 30 and one or two or more relatively thicker struts 32 at or near the top portion of the frame 16 (FIG. 4). The illustrated struts 32 can have a curved configuration, with a concave side facing laterally away from the central medial portion of the frame 16.

The exemplary goggle illustrated herein includes a passive ventilation system as will be apparent from the foregoing. Although the peel away membrane stack of the present invention may be utilized in a goggle without separate ventilation capabilities, the present inventors contemplate the use of the membrane stack in combination with another anti-fogging technology. By allowing passive ventilation of at least some of the accumulated humidity within the entrapped airspace, the effective life of the membrane stack will be extended. In addition, not all activities produce steady state environmental conditions. In the case of downhill skiing or snowboarding, for example, conventional ventilation systems are normally adequate during the rapid downhill descent. However, when the wearer is waiting in a lift line, for example, or otherwise loses forward speed, an air stream is no longer forced into the forward facing vents 38 by the downhill velocity and humidity may be accumulating at a relatively high rate. This confluence creates a relative peak in fogging conditions, which can overcome the rate of air transport via passive ventilation and fogging will occur. At that point, the wearer may choose to remove the goggle and eventually peel away an outer (posterior) most layer 100 to restore a clear field of view.

The illustrated double frame structure thus can define relatively open lateral portions covered by lateral filter membranes 34 (FIG. 3) and a relatively open top portion covered by a top membrane 36 (FIG. 4) between the struts 32. Each of these membranes 34 and 36 preferably can comprise a thin, air permeable material which can keep particles of snow or ice out, while allowing water vapor to pass through. An exemplary membrane material comprises an open cell polyurethane foam.

Referring again to FIGS. 1 to 3, at the bottom portion of the frame 16, laterally on either side of the medial recess 20, the frame can include a pair of front air intakes or vents 38. Each front vent 38 can comprises a passage open at the front of the frame 16, extending between the front frame portion 24 and the rear frame portion 26, and opening upwardly into the entrapped air space defined between the lens and the wearer's face. The illustrated front vents 38 can each include a plurality of slots 39 opening upwardly from the interior of the vents. In some configurations, the illustrated vents 38 can extend from a relatively wide opening at the front of the frame 16 to a relatively more narrow rear portion.

The vents 38, including the slots 39, can be configured to direct air laterally and upwardly into an enclosed space defined between the inner lens 14 (FIG. 6) and wearer's face in the as-worn condition. This configuration causes a circular or other air flow pattern before the air is exhausted through one of the membranes 34 and 36. It has been found that this circulation reduces lens fogging in operation. While not illustrated, each vent 38 preferably includes an air permeable plug (comprising, e.g., polyurethane foam) to prevent ice particles and the like from entering through the vent 38 while dampening air flow to a desired level.

The rear surface of the rear frame portion 26 is preferably lined with a cushioning material 40. As best seen from the cross-sectional view of FIGS. 6, the cushioning material 40 may comprise an inner layer 41, an intermediate layer 42, and an outer layer 43. The inner layer 41 may comprise a soft, matted or woven fiber to prevent chaffing the wearer's face. The intermediate layer preferably comprises a resilient, deformable material such as an open cell polyurethane foam, while the outer layer 42 preferably can comprise a relatively more dense foam material which is readily adhered to the material of the rear frame portion 26.

The exemplary double frame structure shown in FIGS. 1-6 can define an enclosed space or cavity 29 within goggle 10 relatively safe from particulate entry while permitting some ventilation of moisture from this enclosed space. However, under some conditions, moisture can still form on the posterior surface of lens 14.

Referring to FIG. 6, at least one membrane 100 can be positioned adjacent the posterior side of lens 14, and facing the user when goggle 10 is in an as-worn orientation on the user's head. Preferably, a plurality of membranes 100 can be positioned successively relative to each other, and more preferably, attached (e.g., adhered) to each other, to form stack 110 of membranes 100. Membranes 100 can be removable (e.g., peelable) from each other and thus from stack 110, preferably in a successive manner, for reasons described further herein. The membrane stack 110 can be removably attached to a portion of goggle 10 (e.g., lens 14) in various manners described further herein.

Stack 110 can comprise any number of membranes 100 that preferably do not produce an undesirable level of optical distortion (e.g., blur, refraction, absorption, etc.) along the optical pathway, or thickness, of stack 110. It will be understood that the stack 110 is shown in FIG. 6 with four membranes 100 for illustrative purposes only, and that stack 110 can comprise various quantities of membranes 100, such as between about 2 and 100, generally between about 5 and about 20.

The membrane stacks described herein may be mounted on the goggle, helmet shield, and the like, at the point of manufacture. Alternatively, the membranes and/or stacks can be manufactured as an accessory or replacement part and sold independently without a goggle, helmet shield, etc. Any combination of membrane(s) 100, stack 110, and/or goggle 10 can also be supplied as a kit, wherein the membrane(s) 100, membrane stack 110, and/or the eyewear can be supplied separately and then assembled by the user.

Stack 110 can be positioned on the posterior surface of the lens 14 (e.g., within cavity 29) to form a protective barrier that protects (e.g., covers) some, most, or all the posterior surface of lens 14. In some embodiments, stack 110 seals a portion of lens 14 from humid airflow, to prevent condensation on that portion of lens 14. In this way, stack 110 can be positioned to protect lens 14 from condensation or particulates, such as those passing into cavity 29 from the external environment, and those emitted from the user's body into cavity 29.

Stack 110 can be shaped in various ways to facilitate its positioning within goggle 10 and to function as described further herein. Stack 110 can be an approximately rectangular, circular, elliptical, or any other regular or irregular front elevation shape (e.g., FIG. 2) that is sized to protect, cover or seal at least a portion of the primary optical zone of the lens 14. In a preferred embodiment, stack 110 comprises approximately the same size and/or shape as the exposed rear surface of the lens 14. A handling portion such as a pull tab is preferably formed on or attached to each of membranes 100 extending outwardly from stack 110, described further herein.

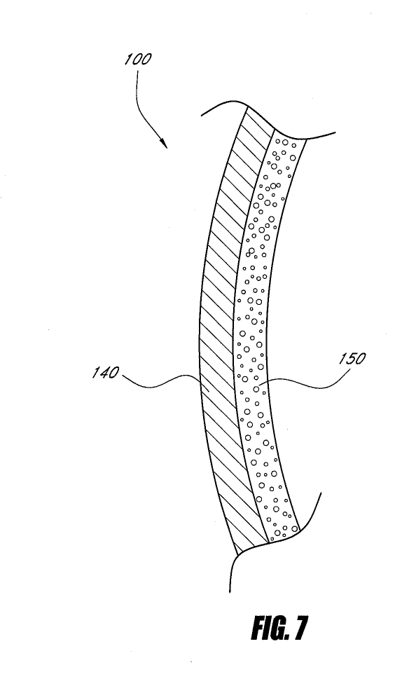

Stack 110 can be formed by pressing, stamping, embossing, die-cutting or punching the approximate shape of stack 110 from two or more sheets of previously stacked layers of rolled or sheet-like material. Alternatively, stack 110 can be formed by pressing, stamping, embossing, die-cutting or punching the approximate shape of membrane 100 from a roll or sheet of material, and subsequently stacking one or more membranes 100 to form stack 110. It will be understood that additional known processing steps, such as thermoforming, mechanical stressing and the like, can be used to pre curve stack 110 (see, e.g., FIG. 10) to conform to a portion of the posterior surface of lens 14. Preforming the stack may not be appropriate for use with planar or cylindrical lenses, but may be desirable for spherical, toroidal or other lenses which are curved about more than one axis.

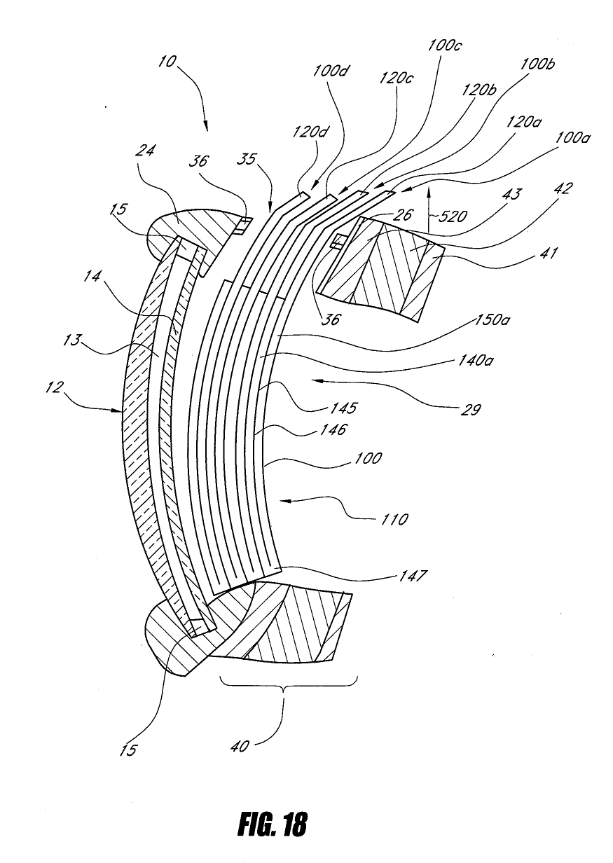

The front elevational shape of membrane 100 can be substantially similar to the front elevational shape of stack 110 described herein. Referring to FIGS. 2 and 17, the height H₁ of the bridge portion of membrane 100 above medial recess 20 and below an upper portion of frame 16 (e.g., when membrane 100 is on viewing window 18) is preferably between about 1.5 inches and 2.5 inches, and is about 2.0 inches for the illustrated embodiment. The height H₂ of the optical zone of membrane 100 on either lateral side of the recess 20 is preferably between about 2.0 inches and 4.0 inches, to provide a greater vertical range of vision to each of the wearer's eyes, and is about 2.7 inches for the illustrated embodiment. The width W of the optical zone of membrane 100 extending from the optical centerline to the outer edge of membrane 100 is preferably between about 1.0 inches and 4.0 inches, to provide a greater horizontal range of vision to each of the wearer's eyes, and is about 2.5 inches for the illustrated embodiment.

Membrane 100 can be attached to another adjacent membrane 100, or to lens 14 using any of a variety or combination of static charge, bonding (e.g., ultrasonic, chemical, thermal, etc.), adhesive (e.g., pressure sensitive adhesive), or other known attachment techniques. Preferably, membrane 100 is attached to an adjacent surface in a manner that allows membrane 100 to be easily peelable therefrom. In some embodiments, a low-tack adhesive or low-strength bonding technique is used.

An attachment region between two adjacent membranes 100 can equal the total surface area of contact between the adjacent surfaces. Alternatively, the adhesive can be applied to no more than about 75% of the contact area, sometimes no more than about 50% and in some configurations no more than about 10% of the contact area. The adhesive can be arranged in strips that can span some, most or all of the length, width, or perimeter of membrane 100, in a continuous or discontinuous manner. The adhesive can be arranged in discrete spots, spaced apart on the contact surface, or spots or strips positioned outside of the primary straight ahead viewing axis through the lens.

In some embodiments, membranes 100 can be attached relative to each other in a manner that helps the stack to substantially conform to the shape or profile of lens 14 or a face shield, etc. (see FIG. 10). For example, tension can be selectively applied to a portion of a first membrane 100 to flex or stretch the membrane as it is being attached to an adjacent membrane held in a quiescent state. After the first membrane 100 is attached to the adjacent membrane, and upon release of the tension, the differential in lateral stress between the two membranes may cause them to curve or flex to a desired profile.

In some embodiments, membrane 100 can be attached to an adjacent element in a manner that minimizes optical interference through membrane 100 and stack 110. For example, membrane 100 may be attached to an adjacent element around a portion of the perimeter of membrane 100, but be unattached within the central viewing zone of membrane 100. Membranes 100 are generally attached to each other and/or lens 14 to the extent that membranes 100 will not peel during use of goggle 10, but can be easily peeled by a user. In some embodiments, membrane 100 can comprise a pull tab 120 that can be grasped by a user when peeling the membrane 100 from stack 110, as described further herein (FIGS. 6, 8, 16-18).

Referring to FIG. 6, stack 110 is shown for exemplary purposes with an optional adhesive layer 130 on a surface (e.g., the anterior-facing surface) of one or more membranes 100. Adhesive layer 130 can be used to attach membranes 100 to each other, to the stack 110, and/or the posterior surface of lens 14. The adhesive layer 130 can comprise oil-based or other adhesives known in the art, and may be a water-based acrylic adhesive. The adhesive layer 130 is preferably highly transmissive in the visible spectrum, although it can be colored, tinted, opaque or translucent to varying degrees. Adhesive layer 130 is shown in FIG. 6 as comprising a distinct layer with a substantial thickness relative to membrane 100, but preferably, the thickness of adhesive layer 130 is negligible once membranes 100 are sequentially laminated or otherwise bonded to form stack 110.

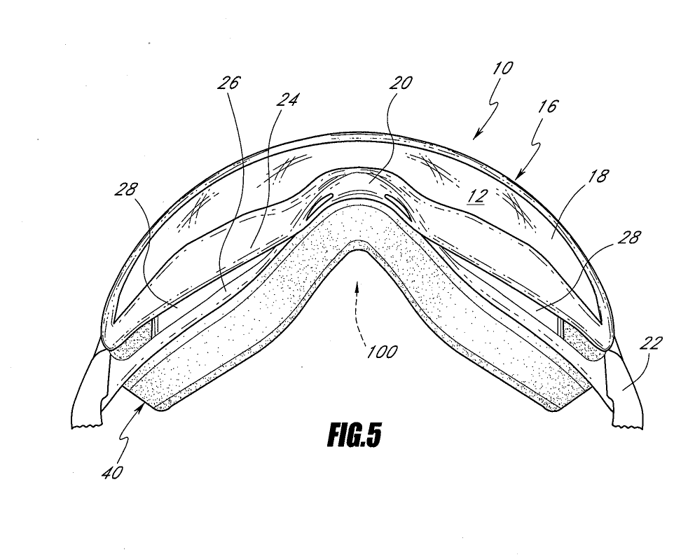

FIG. 7 shows a close-up, partial side cross-sectional view of an embodiment of a single membrane 100 shown in FIG. 6. Membrane 100 can comprise any of a variety of materials and may be a plastic (e.g., thermoplastic) film or membrane. Membrane 100 can comprise a material with a low refractive and/or reflective index, to prevent or reduce optical interference through stack 110. In some embodiments, membrane 100 may comprise a material with a reflective index sufficient to substantially reflect light from stack 110, similar to known reflective lenses in the art. Membrane 100 can be substantially rigid, but is preferably sufficiently flexible to allow it to be easily removed (e.g., peeled) from stack 110.

Membrane 100 can comprise a hydrophobic, hydrophilic, permeable, or impermeable material. In some embodiments, membrane 100 comprises a hydrophilic material to absorb moisture and delay the onset of condensation on membrane 100. Membrane 100 can be clear, translucent or opaque to any degree, and preferably is substantially transparent to allow a user to see through goggle 10. Membrane 100 can be any color, and/or may be printed, for example with brand identification, aesthetic decorations and the like. Membrane 100 can be tinted, polarized or otherwise treated to provide various properties known or described herein along any or all of a portion of its surface, length, width, thickness or perimeter, using various methods known in the art. Membrane 100 can be tinted and/or polarized, such that the overall tint and/or polarization of stack 110 varies, depending on the number of tinted and/or polarized membranes 100 stacked within stack 110. For afternoon skiing, for example, a stack of lightly tinted membranes may start out with a relatively low net transmittance so that it functions as a sunglass. As each membrane is peeled off, the net transmittance of the remaining stack increases. As evening approaches, the net transmittance can approach 100% following removal of all tinted membranes.

The thickness of membrane 100 can be selected based upon its desired physical properties (e.g., flexibility, absorptive capacity, transparency, refractivity, polarity, etc). In some embodiments, membrane 100 can comprise a thickness ranging from approximately 0.0005 to 0.2 inches, generally from approximately 0.001 to 0.1 inches, and in some implementations, from approximately 0.002 to 0.05 inches. In one embodiment, membrane 100 can comprise a thickness of approximately 0.005 inches.

Membrane 100. can comprise two or more layers of materials, or zones within a single layer, illustrated in FIG. 7 as an anterior zone 140 and a posterior zone 150. In the exemplary embodiment, anterior zone 140 and posterior zone 150 are substantially equal in surface area, and span the approximate length and width of membrane 100, although anterior zone 140 and/or posterior zone 150 can span some, most, or all of the length and/or width of membrane 100, and can comprise separate, spaced portions or substantially contiguous portions. Anterior zone 140 and posterior zone 150 can comprise separate layers attached to each other, preferably with a technique which yields greater strength than the attachment between two adjacent membranes 100, to prevent zones 140, 150 from delaminating when membrane 100 is peeled from stack 110. In some embodiments, layers 140, 150 are unattached to each other, or are attached to each other with less strength than the attachment between adjacent membranes 100, to allow zones 140, 150 to move (e.g. slide) relative to each other as membrane 100 is being peeled from stack 110, as described further herein (FIG. 18).

Zones 140 and/or 150 can comprise any of the materials and characteristics of membrane 100 described herein, and can comprise substantially the same or different materials and characteristics relative to each other. Zones 140 and 150 can be formed on a single layer of material that is treated or coated to form two substantially different zones having different characteristics. For example, one or both sides of the acetate membrane can be provided with a surface treatment that causes that side to absorb water, while the other side of the membrane exhibits little or no water uptake.

Zone 150 can comprise a permeable, hydrophilic material to absorb moisture within goggles 10 (e.g., within cavity 29) and delay the onset of condensation forming on lens 14 or stack 110 (FIG. 6). Various types of thin, flexible, hydrophilic and substantially transparent materials (e.g., films or membranes) can be used for at least posterior zone 150, which will be directly exposed to humid air within the goggle and optionally also on an anterior surface of the membrane. These include, for example, water-absorbing polysiloxane, polyurethane wetting treatment, permanent anti-fog coating regenerated cellulose, and treated cellulose acetate. Zone 140 can comprise an impermeable, hydrophobic material to prevent moisture from wicking through membrane 100 and contaminating an adjacent membrane 100. Various types of thin, flexible and substantially transparent hydrophobic materials can be used for zone 140 known in the art, such as a polyester, polyurethane, polyethylene terephalate, or untreated cellulose acetate. In use, zone 150 on the posterior-most membrane 100 in stack 110 can absorb water, and become fully saturated, while zone 140 on the same membrane 100 prevents moisture from wicking into and contaminating the remaining membranes 100 in stack 110. The posterior-most membrane 100 can then be peeled from stack 110, exposing a clean, unsaturated zone 150 on a new membrane 100.

The thickness of zones 140 and/or 150 can be selected for similar reasons as those described herein for the thickness of membrane 100. In an embodiment wherein zone 150 comprises a hydrophilic material, there is a balance between increasing the layer thickness (i.e., to increase absorptive capacity), and decreasing the layer thickness (i.e., to reduce optical distortion or interference through membrane 100). The thickness of zone 140 may be less than the thickness of zone 150, as the hydrophobic material may not require as thick a material to prevent the flow of moisture through layer 140. In some embodiments, zone 150 can comprise a thickness ranging from approximately 0.001 to 0.1 inches, and often, from approximately 0.002 to 0.05 inches, and in some embodiments 0.002 to 0.007 inches. In some embodiments, zone 140 can comprise a thickness ranging from approximately 0.0007 to 0.1 inches, and in some embodiments, from approximately 0.0008 to 0.05 inches, and even more narrowly, from approximately 0.001 to 0.005 inches.

Stack 110 can be attached to various portions of goggle 10 (e.g., to frame portions 24, 26 and/or lens 14) in many different ways, such as those disclosed in U.S. Pat. Nos. 4,076,373; 4,455,689; 4,563,065; 5,592,698; and 4,138,756, the content of which are hereby incorporated herein by reference in their entirety. In some embodiments, stack 110 can be attached to lens 14 by applying the anterior-facing surface of the anterior-most membrane 100 to the posterior surface of lens 14, e.g., by using a pressure sensitive adhesive or any of the techniques described herein for attaching membranes 100 to each other (e.g., FIG. 6). In some embodiments, stack 110 can be attached to frame 10 with a press or friction fit between a portion of the outer edges, surfaces, or annulus of stack 110 and a corresponding portion of the inner edges, surfaces, or annulus of frame portions 24 and/or 26 (e.g., FIG. 6). It will be understood that stack 110 can be attached to other portions of goggle 10, and/or can be attached using other mechanical fasteners such as press fit interference fit, snaps, clips or screws. It will also be understood that stack 110 does not necessarily directly contact lens 14, such that stack 110 and lens 14 can be spaced apart from each other. In some embodiments, goggle 10 can include a peripheral sealing device positioned between stack 110 and lens 14, substantially similar to spacing 13 and gasket 15 between lenses 12 and 14.

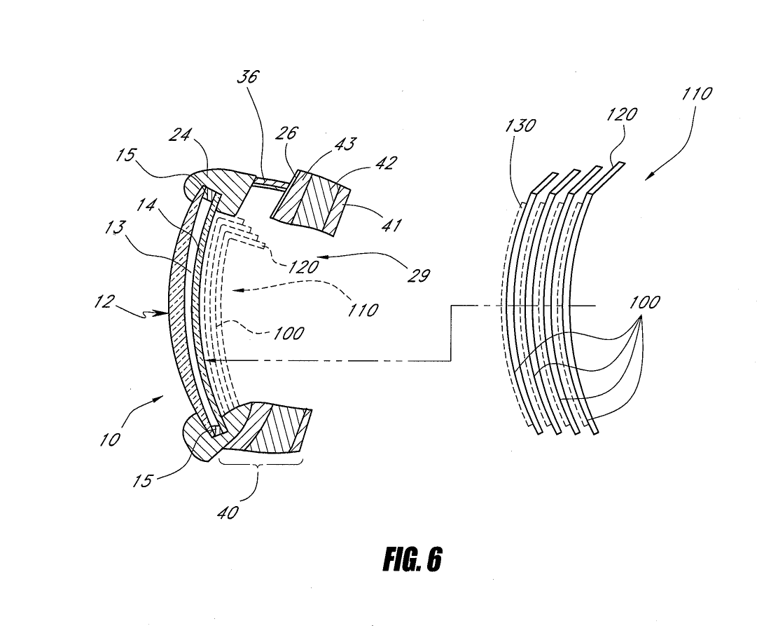

FIG. 8 shows a side cross-sectional and partially exploded view of another embodiment of stack 110 attached to goggle 10. Stack 110 and membranes 100 can comprise one or more openings 115 extending therethrough. Openings 115 can be configured to receive one or more fasteners, such as screws, bolts, rivets, and/or others known in the art, such as exemplary posts 160, attached to goggle 10 within cavity 29. Post 160 can be attached to goggle 10 in any of a variety of ways, such as with threads on post 160 corresponding to a threaded opening in a portion of goggle 10 or adhesives. Post 160 can be extended through a side of one or more portions of goggle 10 (e.g., frame 24, 26, lens 12, 14), and can be secured to an opposite side of said portion with a head, nut, pin, latch, lip or other known fastening element, such as head 161. FIG. 8 shows a lip or flange 24a extending from frame 24 with posts 160 extending through lens 14 and flange 24a for illustrative purposes. Flange 24a can comprise any of many shapes, such as an annular ring-like structure extending inwardly from the perimeter of frame 24, or two or more spaced tabs extending inwardly from the perimeter of frame 24.

Post 160 can comprise a head 162 attached to a proximal end thereof. Head 162 can be configured with a rounded or tapered portion, or other known structure to facilitate advancing the stack 110 to posts 160 in one direction (e.g., an anterior direction, shown by arrow 500). Head 162 can comprise a flatted or shouldered portion, or other known structure to restrict the removal of stack 110 from posts 160 in an opposed direction (e.g., a posterior direction, shown by arrow 510). Post 160 and opening 115 can be configured to restrict the removal of the entire stack 110 from post 160, but to allow a single membrane 100 to be easily peeled from stack 110 and posts 160.

Post 160 can be attached to goggle 10 anywhere that allows stack 110 to be positioned posterior to, and preferably, in sealing engagement with lens 12, as described further herein. Post 160 is preferably attached to goggle 10 proximate to the perimeter of lens 12 in a manner that prevents obstruction of vision through lens 12 and stack 110. It will be understood that posts 160 are shown attached to a portion of goggle 10 proximate to a superior and inferior portion of the perimeter of lens 12 for illustrative purposes only. Post 160 can alternatively or additionally be positioned proximate to a lateral and/or medial portion of the perimeter of lens 12.

FIG. 9 shows a front schematic view of an embodiment of a stack insert 200. FIG. 10 shows a side cross-sectional view of an embodiment of stack insert 200 of FIG. 9 taken along lines 9-9 of FIG. 9. Stack insert 200 can be substantially similar to and function substantially similar to stack 110 as shown in FIGS. 6-8. One difference is that stack insert 200 can comprise a support member 170 that provides additional support to stack 110, to facilitate the peeling of membrane 100 from stack 110, and the insertion and removal of stack insert 200 to and from goggle 10. Support member 170 can be configured to be attached to a portion of goggle 10 and/or to stack 110, using attachment techniques similar to those described herein for attaching stack 110 to goggle 10 (e.g., press fit, fasteners, adhesive, bonding, and the like).

Support member 170 can comprise any of the materials described herein for frame 16, can comprise the same or different material as frame 16, and can be manufactured using similar methods. Support member 170 generally comprises a structure and material with sufficient rigidity to hold and support stack 110 within goggle 10 when goggle 10 is removed from the user's head, and/or when one or more of membranes 100 are peeled from stack 110. Support member 170 comprises a structure and material with sufficient flexibility to allow a user to easily attach stack 110 to goggle 10, and to conform to the flexion of goggle 10 during use.

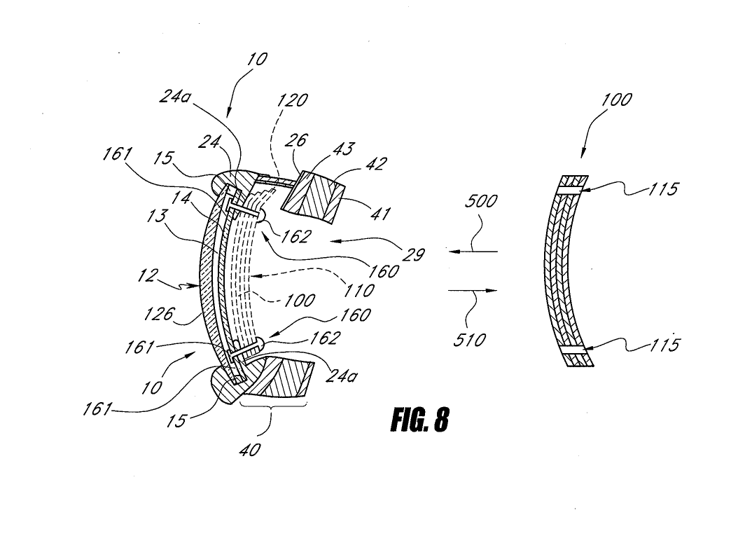

Support member 170 can hold and support a stack 110 of membranes through any of many different configurations. In some embodiments, support member 170 can comprise a sidewall 171 (FIGS. 10, 12, 13) that spans some, most, or all of the perimeter of stack 110. In the exemplary embodiment shown in FIG. 10, sidewall 171 is configured to wrap around, or surround the entire perimeter of stack 110, to hold and support stack 110 within support member 170. In some embodiments, support member 170 can comprise a base 172, a base portion 174 and/or other configurations to support stack 110, as described further herein (FIGS. 11-13).

FIG. 11 shows a partial side cross-sectional view of another embodiment of a stack 210 with many components that are substantially similar to and function substantially similarly to insert 110. In this embodiment, membrane 100 can comprise a membrane support section 100a, a releasable membrane section 100b, and a division 100c between sections 100a and 100b. Releasable section 100b can be configured to be separable from membrane support section 100a at division 100c. Division 100c can be positioned at a distance from the outer perimeter of membrane 100, and can span around some, most or all the perimeter of membrane 100, continuously or in spaced portions. Division 100c can comprise perforations or cuts through membrane 100 that allow releasable section 100b to be peeled from the remainder of stack 110 (e.g., stack support section 110a), without peeling membrane support section 100a from stack 110.

When membranes 100 are stacked to form stack 210, membrane support section 100a and releasable membrane sections 100b can form a stack support section 110a and a releasable stack section 110b, respectively. Stacked membrane support section 110a can be configured to have a greater strength and/or rigidity relative to that of releasable stack section 110b, to facilitate the attachment and removal of stack 210 to and from goggle 10, and to thus function substantially similar to support member 170. In some embodiments, section 110a can comprise a material that is substantially stronger or more rigid than section 110b. In some embodiments, adjacent membrane support sections 100a can be treated, or attached to each other with a stronger adhesive or bonding technique, such that section 110a is substantially stronger or more rigid than section 110b. In this manner, stacked membrane support section 110a can provide additional support to stack 210 during the attachment and removal of stack 210 to and from goggle 10.

FIG. 12 shows a partial side cross-sectional view of a stack insert 220 with many components that are similar to and function substantially similarly to those of inserts 200 and 210. In this embodiment, supporting member 170 can comprise a base 172 attached to sidewall 171. Sidewall 171 and base 172 can comprise any of the materials described herein for member 170, and can comprise the same or different materials relative to each other. Preferably, base 170 comprises a transparent material.

Base 172 can be configured to span some, most or all the length and/or width of stack 110, and/or can comprise one or more individual sections extending from a portion of sidewall 171. In the exemplary embodiment shown in FIG. 12, base 172 is configured within the perimeter formed by sidewall 171, and approximately spans the length and width of stack 110. Base 172 can extend from sidewall 171 at various angles, and is shown for exemplary purposes as extending approximately orthogonal to sidewall 171. Base 172 can be any of many different configurations that provide support to the base of stack 110.

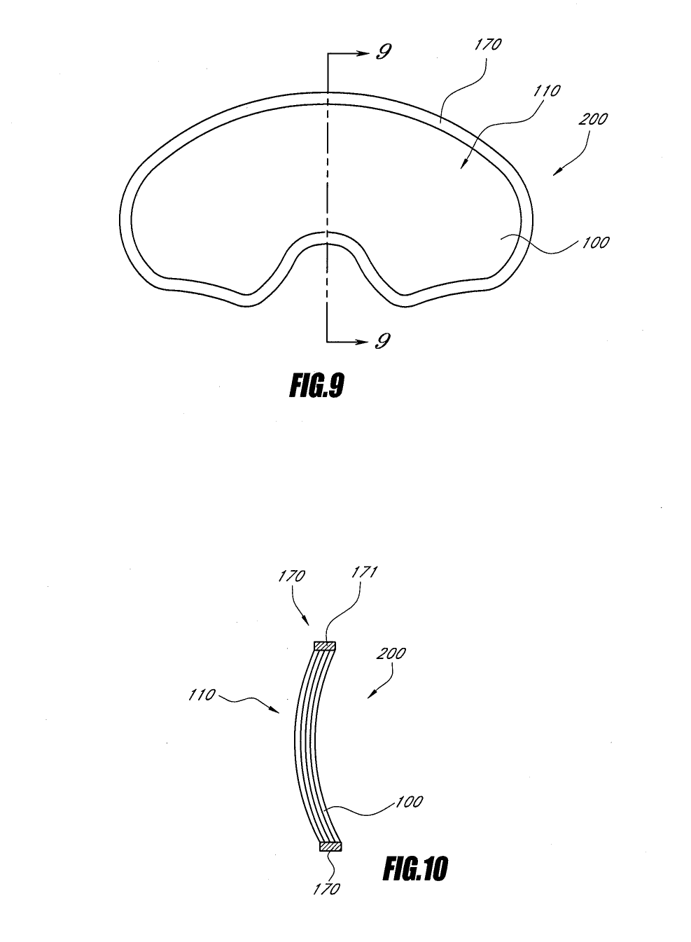

FIG. 13 shows a partial side cross-sectional view of a stack insert 230 with many components that are similar to and function substantially similarly to those of inserts 200, 210 and 220. In this exemplary embodiment, supporting member 170 can comprise a base portion 174 extending inwardly from sidewall 171 (e.g., proximate to an anterior edge of sidewall 171) without spanning across at least one of the width and/or length of stack 110, to support a portion of the outer perimeter of the base of stack 110.

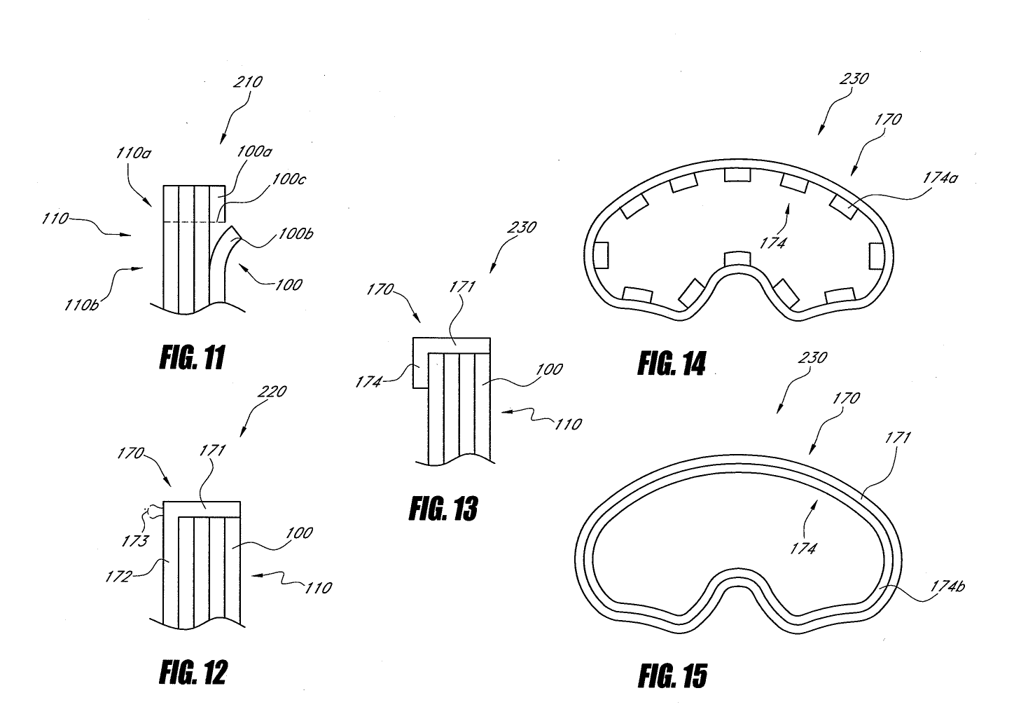

FIG. 14 shows a front elevational view of an embodiment of stack insert 230, with stack 110 removed for clarity. In this embodiment, base portion 174 comprises one or two or four or more tabs 174a extending inwardly from sidewall 171 and spaced apart around the inner perimeter of sidewall 171 to retain and provide an interference fit with a stack 110.

FIG. 15 shows a front elevational view of an embodiment of stack insert 230, with stack 110 removed for clarity. In this embodiment, base portion 174 comprises an annular shelf 174b extending inwardly from sidewall 171 and spanning around the inner perimeter of sidewall 171 to support a stack 110.

The stack inserts shown in FIGS. 9-15 can be attached to a portion of goggle 10 substantially similarly as the attachment of stack 110 to a portion of goggle 110 described further herein (FIGS. 6, 8). In some embodiments of use, support member 170 can be attached to a portion of goggle 10 alternatively or additionally to attaching stack 110 to goggle 10. In some embodiments, support member 170 can be attached to goggle 10 with a press or friction fit. In some embodiments, support member 170 can comprise an opening that receives a fastener attached to goggle 10, functioning substantially similar to opening 115 on stack 110 (FIG. 8). In some embodiments, support member 170 can comprise an attachment element 173 (FIG. 12) that can be configured to receive or be received by a corresponding attachment element on goggle 10 (e.g., a stud or other element that presses or snaps with a corresponding opening or other element). Attachment member 173 can comprise a stud with a head substantially similar to stud 160 and head 160a (FIG. 8), configured to be received by an opening in goggle 10. The structure and positioning of attachment member 173 shown in FIG. 12 is for illustrative purposes only. Attachment member 173 can be positioned on any portion of sidewall 171, base 172, and/or stack support section 110a capable of attaching to a corresponding attachment member on goggle 10. In a preferred embodiment, attachment member can be positioned proximate to the perimeter of support member 170 and/or stack 110.

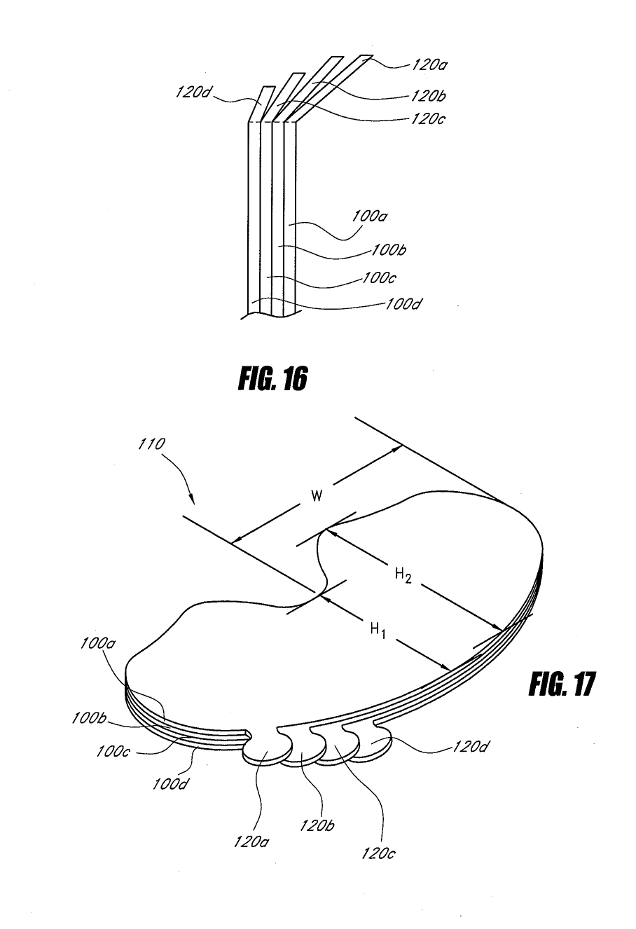

Referring to FIGS. 16-18, as described above, membrane 100 can comprise pull tabs to facilitate a user in peeling membrane 100 from the remainder of stack 110. Pull tab 120 can comprise any of many different known configurations, such as those disclosed in U.S. Pat. Nos. 4,076,373; 4,455,689; 4,563,065; 5,592,698; and 4,138,756.

FIG. 16 illustrates a side view of an embodiment of stack 110. Stack 110 can comprise membranes 100a-100d, illustrated as sequentially positioned in that order relative to each other and in an increasingly anterior direction in that order relative to lens 14 when stack 110 is positioned within goggle 10. Membranes 100a-100d can comprise handling portions (e.g., tabs) 120a-120d, respectively. Pull tabs 120a-120d can comprise any of many different shapes, such as an approximately rectangular, triangular, ovular, mushroom, tear-shaped, semicircular, circular, regular or otherwise irregular shape.

Pull tabs 120a-d can comprise different, or preferably, the same shapes relative to each other. Pull tabs 120a-d can be positioned anywhere along an outer edge of membranes 100a-100d, respectively (e.g., the medial, lateral, superior, and/or inferior edge of membranes 100a-100d). Pull tabs 120a-d can be approximately aligned with each other relative to a line extending approximately perpendicular to the surface through the thickness (e.g., height) of stack 110, or can be spaced from each other relative to such a line (e.g., around a portion of the perimeter of stack 110; FIG. 17). Such spacing can facilitate the removal of one of membranes 100a-100d from stack 110 by a user without simultaneously or inadvertently removing another of membranes 100a-100d. Pull tabs 120a-d can be attached to each other, similar to the remaining portion of membranes 100a-d, or can be free, or unattached relative to each other, as shown for exemplary purposes. Pull tabs 120a-d can be of various lengths relative to each other (e.g., with a sequentially decreasing length as shown), to facilitate the individual grasping, then peeling, of the outermost (e.g., posterior-most) membrane from stack 110.

FIG. 17 illustrates a rear top perspective view of an embodiment of stack 110. Stack 110 can comprise membranes 100a-100d, which comprise pull tabs 120a-120d, respectively, which are substantially similar to and function substantially similar to pull tabs 120a-d in FIG. 16. Pull tabs 120a-d are shown, for exemplary purposes, with an approximately mushroom shape (e.g., with an enlarged “head” portion and a narrower “trunk” portion). Pull tabs 120a-120d are shown, for exemplary purposes, spaced from each other relative to a line extending approximately orthogonally through the thickness (e.g., height) of stack 110 (e.g., spaced around a portion of the perimeter of stack 110).

FIG. 18 shows a side cross-sectional view of an embodiment of stack 110 attached to the goggle 10 of FIG. 1 taken along lines 6-6 of FIG. 2. Stack 110 can comprise membranes 100a-100d and pull tabs 120a-120d that are substantially similar to and function substantially similar to the other embodiments of membranes 100a-100d and pull tabs 120a-120d described herein. In this embodiment, membranes 100a-100d can comprise an anterior sheet 140a and a posterior sheet 150a. Sheets 140a and 150a can be substantially similar to and function substantially similar to zones 140 and 150 (FIG. 7). One difference is that the distal edges of sheets 140a and 150a can be attached to each other at a fold or attachment line 147, such that sheets 140a and 150a comprise two folded sheets that together form membrane 100. Sheets 140a and 150a can be moved relative to each other (e.g., slid, peeled or separated) along a separation line 145, when membrane 100a is pulled longitudinally from its proximal end in the direction shown by arrow 520. Sheets 140a and 150a can be attached to each other along separation line 145 (similarly to the other attachment methods described herein for zones 140 and 150), preferably such that the strength of the attachment between sheets 140a and 150a along separation line 145 is less than the strength of the attachment between adjacent membranes (e.g., along a separation line 146 between membrane 100a and 100b). The strength of the attachment between sheets 140a and 150a is preferably less than the attachment between adjacent membranes to prevent the adjacent membrane (e.g. 100b) from peeling from stack 110 when the first membrane (e.g. 100a) is being peeled from stack 110. The configuration in FIG. 18 allows membranes 100a-100d to be longitudinally, and preferably, individually, and more preferably, sequentially, peeled from stack 110 through a side opening 35 in goggle frame 10 in direction 520. This allows a user to remove membranes 100 from stack 110 without removing the goggles from the user's head.

Although the foregoing invention has been described in terms of certain preferred embodiments, other embodiments will become apparent to those of ordinary skill in the art in view of the disclosure herein. Accordingly, the present invention is not intended to be limited by the recitation of preferred embodiments, but is intended to be defined solely by reference to the appended claims.

What is claimed is:

1. An anti-fogging system for goggles, comprising:
a goggle, having a lens with a posterior surface which faces a wearer in the as worn orientation, to define an air space between the posterior surface of the lens and the wearer's face; and
at least one transparent membrane, removably covering at least a portion of the posterior surface, so that condensation accumulating on a posterior surface of the membrane can be cleared by removing the membrane from the lens.

2. An anti-fogging system as in claim 1, comprising a stack of at least about five membranes.

3. An anti-fogging system as in claim 2, further comprising a support for carrying the membranes.

4. An anti-fogging system as in claim 2 wherein the membranes are removably held together.

5. An anti-fogging system as in claim 4, wherein the membranes are removably held together by an adhesive.

6. An anti-fogging system as in claim 1, wherein the membrane comprises acetate.

7. An anti-fogging system as in claim 6, wherein the posterior surface of the membrane has been treated to enhance water absorption.

8. A method of managing fogging in a goggle, comprising the steps of:
providing a goggle, having a lens with a posterior surface which faces a wearer in the as worn orientation, to define an air space between the posterior surface of the lens and the wearer's face, and at least one transparent membrane, removably covering at least a portion of the posterior surface;
wearing the goggle until condensation is visible on the membrane; and
removing the membrane from the goggle to restore vision through the lens.

9. A method of preparing a goggle, comprising the steps of:
providing a goggle having a lens with a posterior surface which faces a wearer in the as worn orientation, to define an air space between the posterior surface of the lens and the wearer's face; and
removably securing at least one transparent membrane on the posterior surface.

10. A method of preparing a goggle as in claim 9, comprising removably securing a stack of at least about five membranes on the posterior surface.

11. A membrane stack, for attachment to the posterior surface of an eyewear lens, comprising:
a stack of at least three optically transmissive membranes releasably held together such that a single membrane may be peeled apart from the adjacent membrane, the stack having a left optical zone configured for placement in a wearer's left eye line of sight, a right optical zone configured for placement in a wearer's right eye line of sight, and a peripheral edge having a concavity for accommodating a nose region of the eyewear.

12. A membrane stack as in claim 11, wherein the membranes are releasably held together by an adhesive.

13. A membrane stack as in claim 11, comprising at least five membranes.

14. A membrane stack as in claim 11, comprising at least ten membranes.

15. A membrane stack as in claim 11, wherein at least one membrane comprises a pull tab.

16. A membrane stack as in claim 11, wherein a plurality of membranes each comprise a pull tab.

17. A membrane stack as in claim 16, wherein each pull tab is offset from an adjacent pull tab, along an edge of the membrane stack.

18. A membrane stack as in claim 11, wherein the membrane stack comprises a preset curvature.

19. A membrane stack as in claim 18, wherein the membrane stack conforms to a portion of the surface of a sphere.

20. A membrane stack as in claim 18, wherein the membrane stack conforms to a portion of the surface of a toroid.

21. A membrane stack as in claim 11 further comprising a support for mounting the stack to a goggle.

22. A goggle, comprising a frame, configured to support a lens in a wearer's field of view and to define an air volume between the lens and the wearer's face in the as worn orientation, a lens supported by the frame and having a posterior side; and a connector for releasably receiving a stack of peel away membranes on the posterior side of the lens.

23. A goggle as in claim 22, comprising two connectors.

24. A goggle as in claim 22, wherein the connector comprises a post for receiving an aperture on the peel away membranes.
